(19) United States
(12) Patent Application Publication
(10) Pub. No.: US 2021/0272702 A1
(43) Pub. Date: Sep. 2, 2021

(54) METHOD AND SYSTEM FOR ASSESSING LIKELIHOOD OF DISEASE TRANSMISSION

(71) Applicant: Hooman HAKAMI, River Hills, WI (US)

(72) Inventor: Hooman HAKAMI, River Hills, WI (US)

(21) Appl. No.: 16/867,751

(22) Filed: May 6, 2020

Related U.S. Application Data
(60) Provisional application No. 62/983,813, filed on Mar. 2, 2020.

(51) Int. Cl. G16H 50/80; G16H 50/20; G06F 17/18; G06F 1/16; H04W 4/029; H04W 4/021; H04W 4/02

(52) U.S. Cl. CPC G16H 50/80; G16H 50/20; G06F 17/18; H04W 4/027; H04W 4/029; H04W 4/021; G06F 1/163

(57) ABSTRACT

A system for determining likelihood of disease transmission is disclosed. The system can be configured to monitor the exchanges among users of a plurality of communication devices (e.g., mobile phones, tablets, etc.) in a network and determine the probability that a user of a communications device may have contracted a contagious disease by coming in close proximity of another individual using his/her own communications device.

### PRIOR APPLICATIONS

This Application claims priority to U.S. Provisional Application No. 62/983,813, filed on Mar. 2, 2020, the teachings of which is incorporated herein by reference in its entirety.

### FIELD

The present disclosure generally relates to methods and systems for assessing likelihood of disease transmission, and more particularly to methods, apparatus, and systems for electronically assessing likelihood of disease transmission in the past, in real-time, or in near real-time.

### BACKGROUND

During outbreak of a life-threatening disease (e.g., an airborne disease, HIV, etc.), identifying individuals who have been infected with the disease or have come in contact with a person infected with the disease can be critical to public health. For example, when faced with a disease pandemic or a potential disease outbreak, it can be very important to determine whether an individual has been infected with the disease, whether an infected individual has come in contact with other individuals in the community, whether any of the other individuals have contracted the diseases, and whether those other infected individuals have come in contact with and potentially infected others in the community.

Public health officials often rely on manual means of information collection (e.g., patient interviews) to determine an individual's risk of disease transmission. For example, physicians often inquire whether a patient has traveled to regions of the world, where an infection is known to be present (e.g., certain regions of China during the 2019-2020 Coronavirus pandemic) and/or whether the patient has come in contact with a potentially sick individual.

In addition, to determine the potential risk of spread of a disease, physicians and public health officials often have to rely on manually re-tracing the steps of an infected person (e.g., whether the infected person used public transportation, and if so, were they sitting near others, whether the infected individual been in contact with others, whether the infected individual used any protective equipment (e.g., face mask when the contagious disease is a respiratory disease, etc.) in order to determine other individuals who also may have been infected. However, manual information collection can often be tedious, time-consuming, and prone to error.

### SUMMARY

In one aspect, a system for determining likelihood of disease transmission is disclosed. The system can be configured to monitor the exchanges among a plurality of communication devices (e.g., mobile phones, tablets, etc.) in a network and determine some information regarding each communications device. For example, the system can be configured to determine a distance between at least one communication device and at least one other communication device in the network at various pre-determined points of time to determine whether the at least one communication device and the at least one other communication device have been in the vicinity of one another. In an event the system determines that a device has been in the vicinity of another device, the system can determine at least one of: the number of times the devices have been in the vicinity of one another, the dates the devices have been in the vicinity of one another, the times the devices have been in the vicinity of one another, and the duration of the times the devices have been in the vicinity of one another. The system can utilize this information to determine the likelihood of transmission of diseases between users (e.g., known users, known owners, etc.) of the communication devices. For example, upon determining that the user of a communications device has contracted a contagious disease, the system can determine whether the communications device belonging to the infected user has been in the vicinity of other communications devices in the system. The system can use this information, along with other information such as information regarding the duration and/or frequency that the communication devices have been in contact with the communications device of the infected user, to determine the probability that other users have contracted the contagious disease.

In another aspect, a system of assessing the likelihood of disease transmission comprises at least one memory operable to store content collected from one or more user devices over a predetermined period of time and at least one processor communicatively coupled to the at least one memory. The processor can be configured to analyze content received from the one or more user devices to determine whether a device belongs to an infected user having a contagious disease, and in an event the device belonging to the infected user having the contagious disease is identified, receive from the infected user authorization to obtain information regarding user devices which have been in a predetermined proximity of the user. The processor can further analyze the information to determine a probability that a user of each user device which has been in the predetermined proximity of the infected user has contracted the contagious disease, and in an event the probability that the user has contracted the contagious disease exceeds a predetermined threshold, inform the user and/or public health officials of possibility of infection.

In yet another aspect, a system of assessing the likelihood of disease transmission comprises at least one memory operable to store content collected from one or more communications devices over a predetermined period of time, and at least one processor communicatively coupled to the at least one memory. The processor can be operable to collect information indicating whether a first individual carrying a first communications device has been within a predetermined range of one or more individuals carrying respective communications devices, receive information that the first individual is a confirmed carrier of a contagious disease, determine a probability that at least one individual from the one or more individuals has contracted the contagious disease, and inform the at least one individual of the probability that they have contracted the contagious disease.

In another aspect, a system of assessing the likelihood of disease transmission comprises at least one memory operable to store content collected from one or more communications devices over a predetermined period of time, and at least one processor communicatively coupled to the at least one memory. The processor can be operable to collect information indicating whether a first individual carrying a first communications device has been within a predetermined location. The processor can also collect information from communication devices belonging to other individuals entering the predetermined location, receive information that the first individual is a confirmed carrier of a contagious disease, determine a probability that at least one individual from the other individuals has contracted the contagious disease, and inform the at least one individual of the probability that they have contracted the contagious disease.

In other examples, the aspects above, or any system, method, apparatus described herein can include one or more of the following features.

The system can use the information obtained regarding the proximity of the plurality of devices and/or dates and times the plurality of the devices have been in the vicinity of one another to develop a probability map of candidates who appear to have been exposed to an individual known or suspected to have been infected by a disease. The probability map can provide clinician with information that can indicate whether an individual has been in contact with an individual (“sick person”) known or suspected to have been infected by a disease and the possibility that the individual could have been infected by the sick person. In addition, the probability map can provide relevant third parties (e.g., public health officials) with focus (“hot spot”) areas that are that increased risk of outbreak or disease spread.

The predetermined range monitored by the system can be a range in which contagions released from body of the first individual are dispersed. For example, the predetermined range can be at least six feet.

In some embodiments, the one or more communications devices can comprise mobile phones. Further, the processor can be configured to determine the probability as a function of number of times that the at least one individual has been within the predetermined range of the first individual. Additionally or alternatively, the processor can be configured to determine the probability as a function of a length of time that the at least one individual has been within the predetermined range of the first individual. The geographical location can comprise geographical coordinates (e.g., latitude and longitude) of each tracked individual.

Further, the system can be configured to collect the information over a predetermined period of time, for example at least fourteen days. The information can comprise a phone number of each individual and/or a geographical location of each individual.

The system can further comprise a wearable device configured to determine a probability that the contagions are released from the body of the first individuals. The wearable device can comprise at least one of a motion sensor and an audio sensor. Alternatively or additionally, the wearable device can comprise at least one of a thermometer and a heart rate monitor. In some embodiments, the processor can be further operable to confirm whether the first individual is a confirmed carrier of the contagious disease based on information received from the wearable device.

### BRIEF DESCRIPTION OF THE DRAWINGS

A detailed description of various embodiments is provided herein below with reference, by way of example, to the following drawings. It will be understood that the drawings are exemplary only and that all reference to the drawings is made for the purpose of illustration only, and is not intended to limit the scope of the embodiments described herein below in any way. For convenience, reference numerals may also be repeated (with or without an offset) throughout the figures to indicate analogous components or features.

FIG. 1A depicts a high-level block diagram of a system for assessing likelihood of disease transmission according to some embodiments disclosed herein.

FIG. 1B depicts a high-level diagram of possible interconnections among the communications devices in a network monitored by a system according to embodiments disclosed herein.

FIG. 1C is a high-level diagram of a system according to some embodiments disclosed herein.

FIG. 2 depicts a block diagram of a system for assessing likelihood of disease transmission according to some embodiments disclosed herein.

FIG. 3 is a high-level block diagram of digital electronic circuitry and hardware that can be used with, in system for assessing likelihood of disease transmission according to some embodiments disclosed herein.

FIG. 4 is a flow diagram of procedures for assessing likelihood of disease transmission according to some embodiments disclosed herein.

### DETAILED DESCRIPTION

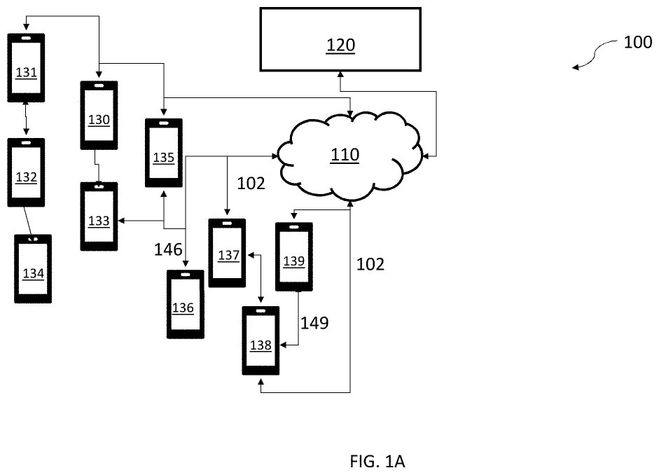

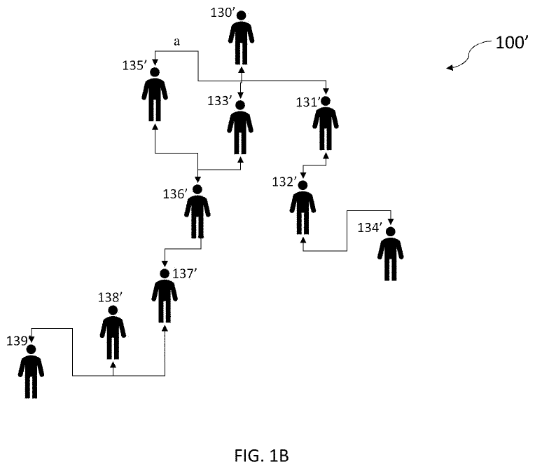

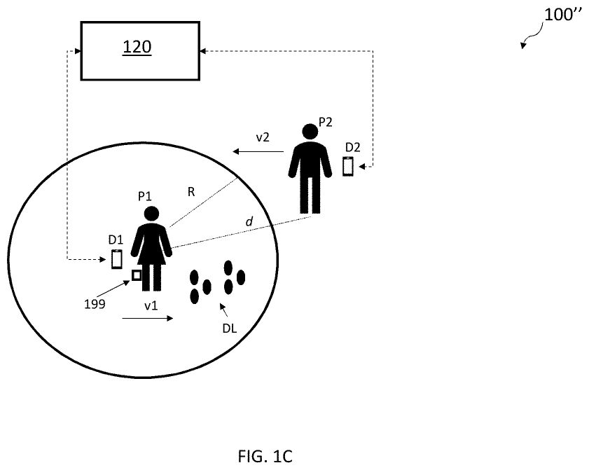

The present disclosure relates to methods and corresponding systems for establishing a temporal pattern of proximity between a user device and other user devices. As shown in FIGS. 1A-1C, in one aspect, the present disclosure relates to a secure system that can utilize user device-to-user device communication to enable healthcare professionals to electronically assess the likelihood of airborne disease transmission in real-time or near real-time. For example, the secure system disclosed herein can utilize existing technology, such as Bluetooth®, Wi-Fi®, Near-Field Communication (NFC) or other communications protocols, to enable a user device-to-user device information exchange that obtains certain information regarding each device and users corresponding to each device. The obtained information can be used to calculate various parameters, such as distance between the user devices (e.g., range), dates, times, and time durations the devices are within a certain range of each other. These parameters can be used to determine various information regarding the users, for example information for assessing a potential likelihood of disease (e.g., airborne diseases) transmission among individual users of the system.

The system can aggregate and utilize the information to develop a probability map of candidates who may have contracted the disease from a disease host or carrier. The system can further alert health authorities and allow relevant authorities to utilize this information to identify potential individuals at risk in the event of a disease outbreak or pandemic. The information obtained from the secure system can further be used to identify those who may have been infected with the disease and/or those who can potentially spread the disease to others. Additionally, this information can provide public health officials with “hot spot” areas that are at a higher risk of outbreak or disease spread.

In a related aspect, a method for establishing a temporal pattern of proximity between a user device and other users devices is disclosed. The disclosed method monitors a plurality of communication devices in a system and determines whether a communication device belonging to a user of the system is within a communication range of one or more other communication devices belonging to other users of the system. The communication range may vary based on the type of communications protocol employed to provide a communication channel between the devices. Using existing technology, such as Bluetooth®, Wi-Fi®, Near-Field Communication (NFC) or other communications protocols a communications channel can be established between the user's communications device and the other communication devices. The communications channel can be used to obtain certain information regarding the users of the other communications devices. Such information can include, for example, the phone number, device serial number, distance between the devices and the time the two devices were able to maintain communication with each other. The information obtained/collected from the users of the other communications devices can be stored in a memory (e.g., memory of a user device) or transmitted, via a network (e.g., the Internet) to a server to be stored on the server.

The communications range can be any suitable range. For example, as shown in FIG. 1C, droplets DL generated by infected individuals P1 carrying certain respiratory diseases are known to travel as far as six feet from an infected individual. Therefore, for monitoring possible community transmission of such diseases, the system 100″ can monitor the communications devices D1, D2 in the system to determine whether they have been within six feet of one another. As noted this range R can be any suitable range and vary for various contagious diseases. For example, in monitoring possibility of transmission of HIV, this range can be a smaller distance. The data corresponding to the temporal pattern of proximity can be used, for example, to assess the likelihood that the user P2 may have been infected with a disease due to his/her proximity of a person P1 carrying that disease.

In addition to range and proximity of the communications devices, the system can consider other factors such as duration of exposure. For example, in some embodiments, the system can consider whether an individual P2 and his/her communications device D2 were in the close proximity of an infected individual P1 and his/her communication device D1 for a certain period of time (e.g., 5, 10, or 15 minutes). Alternatively or additionally, the system can consider the frequency of the contacts between the infected person and others. For example, the system can consider the number of times an infected individual P1 has been in contact with other individuals P2 (e.g., infected person and another person have been within two feet of each other seven times in the three days).

The system can also be configured to monitor and record the interactions between the communications devices over a predetermined time span. For example, for a respiratory disease having an incubation period ranging from about two days to about fourteen days, the system can be configured to record and monitor interactions between individuals for at least fourteen days (e.g., fourteen days prior to identifying a person P1 as having been infected with the contagious disease). Generally, any suitable time span can be used with the embodiments disclosed herein.

FIGS. 1A-1C depict high-level block diagrams of system 100, 100″ for assessing likelihood of disease transmission according to some embodiments disclosed herein. The systems 100, 100″ can comprise a server 120 that connects, via a communications network 110, to a plurality of communication or user devices D1, D2/131, 132, 133, 134, 135, 136, 137, 138, 139. The communication devices can be coupled to the network via one or more links 102. The user devices 131, . . . , 139 can be interconnected and be in communications with one another. Specifically, these devices can be configured such that they connect to each other via the communications network 110. Alternatively, or additionally, the user devices can be configured such that they are directly connected to each other, via one or more link 146, 149.

Generally, any suitable communication protocol and/or communication link can be used to couple the user devices to the network and/or to each other. For example, wired or wireless links can be used to couple the user devices to the network. Alternatively, or additionally, the communications devices can be connected to each other via the network 100 or any suitable communications protocol, such as Bluetooth®, Wi-Fi®, Near-Field Communication, etc.

Examples of the user devices 131, . . . , 139 that can be used with the embodiments disclosed herein include, but are not limited to, workstations, wireless phones, smart phones, personal digital assistants, desktop computers, laptop computers, tablet computers, handheld computers, smart phones, etc.

The network 110 can generally be any suitable network. For example, the network 110 can be a private network (e.g., local area network (LAN)), a metropolitan area network (MAN), a wide area network (WAN), or a public network (e.g., the Internet). The communications network 110 can also be a hybrid communications network 110 that includes all or parts of other networks. The networks 110 can have various topologies (e.g., bus, star, or ring network topologies).

The server 120 can include any suitable and required circuitry for implementing the procedures described herein. For example, as detailed below, the server can comprise a processor 310, a main memory 329, and a database 330, which are configured to store and execute the procedures described herein. Each communications device can also include suitable and required circuitry for implementing the procedures described herein.

As shown in FIG. 1A, the system can monitor the interactions of any given communications device 130 with other devices in the network. For example, the system can observe that the communications device 130 has been in close proximity (e.g., six feet when tracking a contagious respiratory disease, within the range R) with a number of other devices 131, 133, 135 in the system. Similarly, the system can track and monitor the interactions of these devices 131, 133, 135 with other devices in the system. For example, the system can determine that a device 131, after coming in close proximity of device 130, has come in contact with a number of other devices 132, 134. The system can track, record, and monitor such interconnections with all devices in the network (e.g., contact between devices 133, 135, and 136, contact between devices 136, 133, and 137, etc.).

Upon determining these interconnections, the system can create a table, tree, or any suitable construct (e.g., mathematical construct) for understanding these interconnections. For example, as shown in FIG. 1B, the system can create a table 100′ that links the individual users of the communications devices and describes and track their connections. The system can update this table dynamically, as the users of the systems move about a monitored region and come in contact with other individuals. As shown, the table 100′ demonstrates that an infected person 130′ (operating device 130) has come in contact to and potentially infected person 135′ (operating device 135), person 131′ (operating device 131), and person 133′ (operating device 133). These individuals then have come in contact and potentially infected person 132′ (operating device 132), and person 136′ (operating device 136), who then came in contact with person 134′ (operating device 134) and person 137′ (operating device 137) and potentially infected these individuals. Person 137′ (operating device 137) then came in contact with Person 138′ (operating device 138) and potentially infected this individual, who later came in contact with Person 139′ (operating device 139) and potentially infected this individual 139′. The system can maintain and update any suitable relationship table/chart such as this chart 100′ to determine the connections among the users of the network.

In some embodiments, the system can assign weights to the links (for example, link a) connecting these individuals. The weights can be assigned to the links based on various factors, such as the length of the time the individuals were in proximity of each other, the frequency of contacts (e.g., how many times they had contacts), and other factors (e.g., whether a protective measure, such as a facemask was used). Generally, depending on the disease being tracked, various other factors can be considered in assigning weights to the interactions between the individuals in the network.

It should be noted that the individuals in the network can be individuals who have willingly signed up and registered for monitoring using embodiments disclosed herein. Such individuals can consent/sign up for monitoring using embodiments disclosed herein using any suitable technique known in the art, for example by downloading an application software that carries out the procedures according to embodiments disclosed herein. Additionally or alternatively, the monitoring procedures disclosed herein can be required by the service provider and/or be regulated by governments, which are installed on a user's device using any suitable technique (e.g., included in an update to an operating system or pushed on the user's device by the service provider).

As detailed below, embodiments disclosed herein monitor and record interactions between users of various devices over a predetermined period of time. Upon being notified that a user of a communications device monitored by the system has tested positive for a contagious disease (or is presumed to have contracted a contagious disease), embodiments disclosed herein assign a probability that each member that has come in contact with the infected person has also contracted the disease. A system according to embodiments disclosed herein can be configured to notify those who may have infected the disease based on the probabilities determined by the system. In some embodiments, results obtained from antibody testing can be used to determine whether a person has been a silent carrier of a contagious disease and initiate tracking and notifying his/her contacts.

The system can be notified regarding existence of an infected person (person 130) using any suitable technique. For example, the infected person 130′ can notify the system using an interface of an application software installed on his/her communications device 130. Alternatively or additionally, the system can be notified by healthcare providers, healthcare authorities, and/or government bodies.

Similarly, possibly infected individuals can be notified using any suitable mechanism known in the art. For example, the system, upon determining that a person 133′ may have contracted a disease (i.e., when the probability of the person having contracted the disease is higher than a predetermined value, e.g., probability ≥0.5), can contact the possibly infected person 133′ using any suitable technique, such as a push notification sent through the downloaded application software, a phone call, or a text message. The notification message can request that the possibly infected person 133′ proceed to be tested for the contagious disease (e.g., with information and directions for how to get tested) and report back to the system whether he/she has in fact contracted the contagious disease.

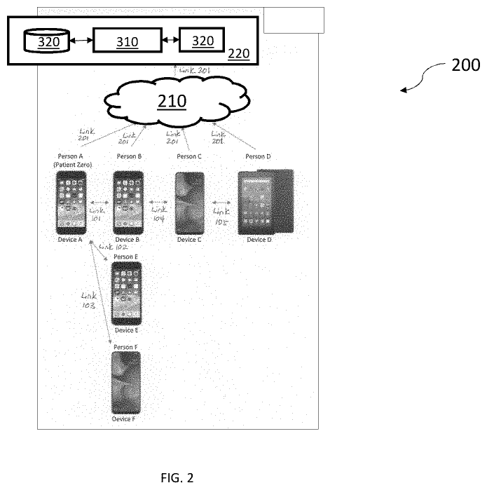

FIG. 2 is a block diagram of a secure device-to-device communication scheme and associated analytics 200 that can enable healthcare professionals to electronically assess the likelihood of airborne disease transmission in real-time or near real-time.

As noted, the device-to-device communications scheme and associated analytics 100 includes a server 220 that is coupled via communications network 210 with a number of communications devices, Device A, Device B, Device C, Device D, Device E, and Device F. Each device can belong to a corresponding user, user A, user B, user C, user D, user E, and user F, respectively. As noted, these devices can be in communications with one another via any suitable communications protocols including, but not limited to, Bluetooth®, Wi-Fi®, Near-Field Communication, etc.

The devices (Device A, Device B, Device C, Device D, Device E, and Device F) can be configured to connect to one another automatically. The devices can be configured such that once connected, they can exchange information relevant for airborne disease transmission analysis, or other analysis that would require information regarding proximity of devices. Such information can include, but is not limited to, device serial number, proximity of one device with another, and the time these devices were within a specific distance (range). This information is stored securely and in an encrypted fashion on the communication devices.

Once a patient is identified as a source of a disease (e.g., an airborne disease) by checking into a hospital and/or testing positive for the disease (e.g., Patient 0, Device A), the pertinent information relevant for airborne disease transmission analysis from his/her corresponding device, Device A, can be directly transmitted to the server 102 through the communications network 210. In some embodiments, the system can be arranged such that the process of connecting the pertinent information relevant for airborne disease transmission analysis from communication Device A to the server 102 can only be done with user permission.

Once a user has granted permission to extract the pertinent information relevant for airborne disease transmission analysis from their communication device, the information is transmitted via the network 210 to the server 220. As noted, transmission any information can be voluntary and at the user's discretion and/or mandated by local, government, or health rules and performed without consent and/or knowledge of the user.

The Application 220 can also include the capability for data storage, data analysis and report generation. For example, the application 220 can include the capability for maintaining the information regarding contacts over a predetermined time. Further, as detailed below, the server 220 can include in any computing device designated to run one or more services or function as host. This can include, but is not limited to, a computer, various servers (e.g., gaming web, mail, etc.), a phone, tablet, etc.

Once the pertinent information relevant for disease transmission analysis from the communication devices has been transmitted to server 220, it can be stored in a data storage facility 320. Such information can include, but is not limited to, device serial number, proximity of one device with another, and the time these devices where within a specific distance (range). The processor 310 can access the data in the data storage facility 320 and use information such as, but not limited to device serial number, proximity, and time within a specific distance (range) to assign a probability of transmission from a potential disease host to individual(s) they may have come into contact with. This probability would be calculated on a disease-by-disease basis and based on guidelines set by public health officials (e.g., CDC, World Health Organization, etc.) that assess the likelihood of transmission. For example, for the novel coronavirus, guidelines from the federal Centers for Disease Control and Prevention define “close contact” as anyone who has been within 6 feet of a person infected with the virus for a “prolonged period of time,” However, for a disease such as the measles, officials broadly publicize every known location an infected person frequented in the days before being diagnosed, and try to track down people who came in contact with that patient who have not been vaccinated.

For example, as shown in FIG. 2, the processor 310 can determine and identify Person B, Person E, and Person F as individuals with whom Patient 0 (Person A) has come into contact. Further, and based on the information about Person B, Person E and Person F, such as, but not limited to, proximity of device B, device E and device F to Person A (device A), and the time these devices were within a specific distance (range) of the device of Patient 0 (Person A), the processor 310 can determine the probability of airborne disease transmission from Patient 0 (Person A) to Person B, Person E, and Person F, respectively. This probability would be disease specific and based on guidelines set by public health officials.

Based on the probabilities of transmission identified, the processor 310 can generate a detailed report that can identify, among other things, the serial numbers of the individuals with whom Patient 0 (Person A) has come into contact, the probability of transmission of the airborne disease from Patient 0 (Person A) to Person B, Person E and Person F, and determine the current location of the individuals with the highest probability of infection.

In some embodiments, the system, can automatically contact the individuals, Person B, Person E, or Person F, with the highest probability of transmission to request permission to test these individuals for the specific disease and/or for conducting the procedures described herein to determine whether other individuals have come in contact with these possibility infected individuals. The system can contact the possibly infected individuals through either manual or electronic means—such as, but not limited to, through an application or via system generated notifications, such as an “Amber Alert” type method.

In some embodiments, one or more of the following scenarios and their associated methods for calculating the probability of infection can be employed. The following abbreviations are used in the discussion below:
P=probability of infection
d=distance between two individuals
t=duration of interaction
v=vector of movement
R=exposure range (This is a constant and unique to each specific disease)
E=minimum incubation period (This is a constant and unique to each specific disease)

For example, referring back to FIG. 1C, a system 100″ according to some embodiments disclosed herein can determine the probability P that an individual P2 can be infected by an infected individual P1 when the first person (P1, also referenced herein as the first patient or Patient 1) is in a distance d of the second person (P2, also referenced herein as the second patient or Patient 2). In the example provided in FIG. 1C, the contagious disease is transmitted via exposure to droplets DL when the second person P2 is within a range R of the first person P1. In this example, the first person P1 is traveling along a first vector v1, while the second person P2 travels along a second vector v2.

In one example, a first patient (P1/Patient 1) can directly infect a second patient (P2/Patient 2). Each communications device D1, D2 (hereinafter “mobile device”) can be configured to continuously, or at least over some predetermined time periods, capture various information regarding any other mobile device with which it comes in contact. The information can include any suitable information, such as distance R of the mobile device D1 from any other device D2, duration of proximity between the mobile device and other mobile devices, and vector data v1, v2 for each new mobile device that comes in a predetermined vicinity of the mobile device.

For example, the mobile device D1 of Patient 1 can be configured to record information such as distance, duration, and/or other information and features relating to other mobile devices that it may come in contact with. An assignment of probability of infection can occur once an exposure range, R, has been defined. For example, once an exposure range for a particular disease has been defined, the probability of Patient 1, P1, infecting Patient 2, P2, is represented with each individual element as follows:

The probability of infection is greater as the distance d within the exposure range decreases between two individuals. Therefore Probability P and Distance d can be inversely related. In some embodiments, such an inverse relationship can be a linear inverse relationship (e.g., P∝1/d). Alternatively or additionally, the inverse relationship can be a non-linear inverse relationship. By way of example, in some cases where the spatial expansion of a pathogen released into environment (e.g., via coughing an infected individual), this range can be assumed to be substantially isotropic and the probability P can be inversely related to the square of d (i.e., P∝1/d²). In some embodiments, the constant of proportionality can be determined via calibration (e.g., experimentally or by simulation). For example, in a controlled environment a sample of a pathogen can be released in an aerosol for and it is propagation can be mapped to determine range of spread.

In some embodiments, different inverse relationships or any other suitable relationship can be employed for different pathogens (obtained via simulation). Further, in some embodiments, the probability P can be set to zero if d is greater than the exposure range R. Also, the probability P can have a finite value when the distance d between two individuals is at or near zero. This finite probability can decrease as a function of d according to a suitable inverse relationship, such as those discussed above.

Further, the probability of infection can become greater as the duration of exposure between two individuals within the exposure range increases. Thus, in some embodiments, the Probability (P) and the duration of interaction (t) can be directly proportional.

Furthermore, in some embodiments, the relative spatial positions for Patient 1 and Patient 2 and/or their relative movements can be taken into account for calculating the probability P. For example, the probability of infection can be greater if the vector of movement for Patient 1 is opposite the vector of movement for Patient 2. In other words, the probability can be greater if the two individuals are moving in opposite directions and, therefore, facing each other while they are in the exposure range.

It should be noted that the correlation between the probability of infection and the spatial distance of the two individuals (i.e., Patient 1 and Patient 2) need not be a direct correlation. Generally, any suitable relationship can be used to describe this relationship. For example, in some embodiments, the dot product of two vectors associated with the velocity of Patient 1 and Patient 2 can be employed to scale the probability P. Such a dot product can be defined as follows:

V1·V2=|V1∥V2|cos(θ),

where |V1| and |V2| are magnitudes of the velocity vectors associated with Patient 1 and Patient 2, respectively, and θ is an angle between these two vectors. In some such embodiments, when the dot product is positive, the probability (P) can be scaled up and when the dot product is negative, P can be scaled down. For example, when the dot product is positive, the probability can be scaled up by a factor proportional to the dot product, and when the dot product is negative, the probability can be scaled down by a factor proportional to the dot product. In some embodiments, the proportionality factors can be obtained empirically for a given pathogen (e.g., in a manner discussed above).

In certain embodiments, the probability of infection can be refined based on considering additional elements. For example, wearable technology 199 can be utilized to determine whether an individual has coughed or sneezed. This can done by using gyroscopes, one or inertial sensor (e.g., to detect motions of a person's arm in response to sneezing or to cover their cough or sneeze), one or more motion sensors, and/or other sensors within the wearable technology 199 to identify and track the hand motion associated with a sneeze or a cough. For example, the sensors can determine whether the person P1 has made any motions that can be associated with sneezing or coughing. Once it has been determined that Patient 1 has coughed or sneezed, this can increase the probability that individuals within exposure range R have been infected. In some embodiments, an audio sensor can be utilized to determine if the person P1 is speaking (since certain respiratory diseases can be communicated through speaking) and/or detect the sounds associated with coughing and sneezing. In addition, this information can decrease the emphasis of the duration of interaction, d, on the probability because once and individual has sneezed or coughed, the chances of infection causing droplets infecting others become higher, irrespective of the time of exposure.

In some embodiments, a diffusion equation, such as the following equation, can be used to estimate the diffusion of aerosols and/or droplets released into the environment via Patient 1's sneeze or cough:

$$\frac{\partial ∅ ( r , t )}{\partial t} = ∇ \cdot [ D ( ∅ , r ) ∇ ∅ ( r , t ) ]$$

where Ø(r, t) is the density of the diffusing material at location r and time t, and D(Ø, r) denotes the diffusion coefficient for density Ø at location r; and ∇ represents the gradient operator. In some embodiments, the diffusion coefficient can be estimated, for example empirically, for a given pathogen. In some embodiments, such a diffusion equation can be used to determine the probability that an individual who was within a range R (for a given time period) of another individual who had sneezed or coughed may have been exposed to the pathogen.

As noted, the system can also utilize wearable technology 199 to determine patients who may have the disease even though they may not have been tested for the disease. By using the information from temperature sensors from the wearable 199, as well as, but not limited to, heart rate variability measurements and trends, embodiments disclosed herein can determine individuals that may have a fever and/or are experiencing deteriorating health. This information can be used to refine the probability of infection, P. Additionally or alternatively, this information can be used to assess the risk of disease spread from asymptomatic and/or untested individuals.

Embodiments disclosed herein can also consider the possibility that Patient 2 can move on to infect another individual, Patient 3, in determining the probability of disease transmission. Once Patient 2 is deemed to be at a high probability of infection, the probability of Patient 2 infecting Patient 3 utilizes the same procedures as those described above with respect to Patient 1 and Patient 2. However, the probability can add the minimum incubation period E variable to the equation. In other words, in some implementations, the probability that Patient 2 infects Patient 3 is determined after the minimum incubation period has passed.

Embodiments disclosed herein can further consider that Patient 1 can indirectly infect Patient 2 into determining the probability of disease transmission. Specifically, embodiments disclosed herein can consider that for some contagions and diseases, Patient 1 and Patient 2 need not to directly come in contact with one another. For example, transmission of Flu and similar respiratory diseases (e.g., Novel Coronavirus) or certain bloodborne diseases (e.g., Hepatitis C) via droplets and/or blood released by the infected person (e.g., droplets released via coughing or sneezing or blood left in a dirty needle, such as a tattoo needle).

In such cases, although Patient 1 and Patient 2 have not come in direct contact with one another, Patient 2 has occupied a space that Patient 1 has also occupied (for example, a seat on an airplane) and potentially infected and/or has been exposed to contagions released from Patient 1 (e.g., Patient 1 is a letter carrier that delivered mail to Patient 2 and a piece of mail carried the contagion from Patient 1's body into Patient 2's house or Patient 2 was tattooed with the same needle as Patient 1). In this case, the probability of Patient 1 exposing Patient 2 can be a function of the duration of contact between the sick individual and the environment and items. Specifically, the probability that patient 1 has infected a particular area or a particular item is a function of the duration that Patient 1 has spent in that particular area without moving or the duration that Patient 1 has come in contact with the item. Generally, the longer Patient 1 has spent in a particular area without moving, the greater the likelihood that Patient 1 has infected that area. Similarly, the longer Patient 1 has come in contact with the item, the greater the likelihood that Patient 1 has infected that item.

Generally, the probability that Patient 2 has become exposed can be a function of two variables: First: Patient 2 must have also occupied the same space or come in contact with same objects as Patient 1. This does not have to necessarily be a perfect match of location and can be within a perimeter of the exposure range defined by the disease. Second: Patient 2 must have spent sufficient time in that particular space to have been exposed. Therefore, the probability that Patient 2 has been infected can be directly proportional to the amount of time Patient 2 has spent in the infected area.

In some embodiments, the probability that Patient 1 has infected Patient 2 can also be a function of whether the contact between Patient 1 and the environment has been a protected or unprotected contact (e.g., if the contagious disease is a respiratory disease, whether Patient 1 was wearing a mask).

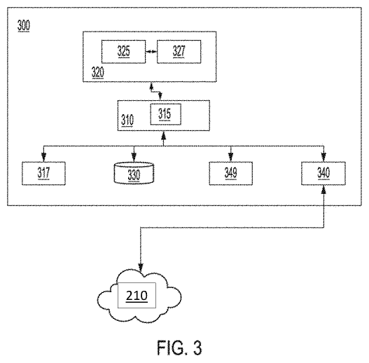

FIG. 3 is a high-level block diagram of digital electronic circuitry and hardware 300 that can be used with the embodiments disclosed herein. The electric circuitry 300 can include a processor 310 that is configured implement the procedures disclosed herein.

The processor 310 can be configured to collect or receive information and data from the user devices and/or store or forward information and data to the user devices and/or another entity (e.g., a healthcare entity, a healthcare provider, etc.). The processor 310 can further be configured to control, monitor, and/or carry out various functions needed for analysis, interpretation, tracking, and reporting of information and data collected by the system for assessing likelihood of disease transmission as disclosed herein. Generally, these functions can be carried out and implemented by any suitable computer system and/or in digital circuitry or computer hardware, and the processor 310 can implement and/or control the various functions and methods described herein.

The processor 310 can further be generally configured to implement procedures for detecting likelihood of disease transmission and/or send and/or receive signals from the various user devices. The processor 310 can also collect or receive data regarding each user device (e.g., location data) and/or store or forward the data to another entity (e.g., a medical facility, etc.).

The processor 310 can be connected to a main memory 320, and comprise a central processing unit (CPU) 315 that includes processing circuitry configured to manipulate instructions received from the main memory 320 and execute various instructions. The CPU 315 can be any suitable processing unit known in the art. For example, the CPU 315 can be a general and/or special purpose microprocessor, such as an application-specific instruction set processor, graphics processing unit, physics processing unit, digital signal processor, image processor, coprocessor, floating-point processor, network processor, and/or any other suitable processor that can be used in a digital computing circuitry. Alternatively or additionally, the processor can comprise at least one of a multi-core processor and a front-end processor.

Generally, the processor 310 and the CPU 315 can be configured to receive instructions and data from the main memory 320 (e.g., a read-only memory or a random access memory or both) and execute the instructions. The instructions and other data can be stored in the main memory 320. The processor 310 and the main memory 320 can be included in or supplemented by special purpose logic circuitry. The main memory 320 can be any suitable form of volatile memory, non-volatile memory, semi-volatile memory, or virtual memory included in machine-readable storage devices suitable for embodying data and computer program instructions. For example, the main memory 320 can comprise magnetic disks (e.g., internal or removable disks), magneto-optical disks, one or more of a semiconductor memory device (e.g., EPROM or EEPROM), flash memory, CD-ROM, and/or DVD-ROM disks.

The main memory 320 can comprise an operating system 325 that is configured to implement various operating system functions. For example, the operating system 325 can be responsible for controlling access to user devices, memory management, and/or implementing various functions of the system for assessing likelihood of disease transmission disclosed herein. Generally, the operating system 325 can be any suitable system software that can manage computer hardware and software resources and provide common services for computer programs.

The main memory 320 can also hold application software 327. For example, the main memory 320 and application software 327 can include various computer executable instructions, application software, and data structures, such as computer executable instructions and data structures that implement various aspects of the embodiments described herein. For example, the main memory 320 and application software 327 can include computer executable instructions, application software, and data structures, such as computer executable instructions and data structures that implement a user interface on a user's device, which can be employed to communicate with the user in order to receive commands from the user (e.g., authorization to share data).

Generally, the functions performed by the system for assessing likelihood of disease transmission 200 can be implemented in digital electronic circuitry or in computer hardware that executes software, firmware, or combinations thereof. The implementation can be as a computer program product (e.g., a computer program tangibly embodied in a non-transitory machine-readable storage device) for execution by or to control the operation of a data processing apparatus (e.g., a computer, a programmable processor, or multiple computers).

The main memory 320 can also be connected to a cache unit (not shown) configured to store copies of the data from the most frequently used main memory 320. The program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network, such as the Internet.

The processor 310 can further be coupled to a database or data storage 330. As noted, the data storage 330 can be configured to store information and data relating to various functions and operations of the system for assessing likelihood of disease transmission 200. For example, the data storage 330 can store the data collected by the server 220 from each device, Device A, Device B, Device C, Device E, in the network.

The processor 310 can further be coupled to a display 317 (which can be the display of the user device). The display 370 can be configured to receive information and instructions from the processor. The display 370 can generally be any suitable display available in the art, for example a Liquid Crystal Display (LCD) or a light emitting diode (LED) display. For example, the display 370 can be a smart and/or touch sensitive display that can receive instructions from a user and/or provide information to the user.

The processor 310 can further be connected to various interfaces. The connection to the various interfaces can be established via a system or an input/output (I/O) interface 349 (e.g., Bluetooth, USB connector, audio interface, FireWire, interface for connecting peripheral devices, etc.). The I/O interface 349 can be directly or indirectly connected to the ophthalmic testing system 150.

The processor 310 can further be coupled to a communication interface 340, such as a network interface. Generally, the communications interface 340 can be a communications interface that is configured to provide the system 200 with a connection to a suitable communications network 210, such as the Internet. Transmission and reception of data, information, and instructions can occur over the communications network 210. Further, in some embodiments, the communications interface 340 can be an interface that is configured to allow communication between the digital circuitry 300 implemented in one user device (e.g., Device A) and another user devices (e.g., Device B) via any suitable communications means, such as a wired or wireless communications protocols including WIFI and Bluetooth communications schemes).

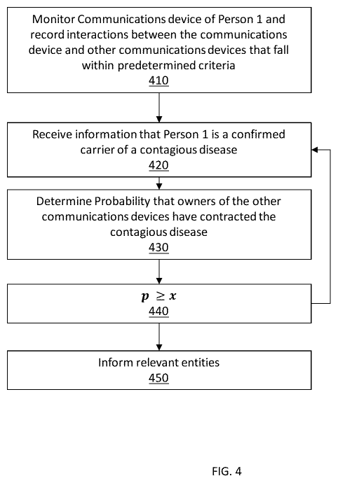

FIG. 4 is a flow diagram of procedures for assessing likelihood of disease transmission according to some embodiments disclosed herein. As shown, a system according to some embodiments disclosed herein can monitor communications devices belonging to each monitored individual in the system and record interactions among the monitored communications devices (box 410). For example, the system can monitor a communications device (D1, FIG. 1C) belonging to a person (P1, FIG. 1C) and record all interactions of the device other devices (D2, FIG. 1C) in the system. The system can record the interactions that fall within predetermined criteria (e.g., when other devices are within a certain range R of the monitored device D1).

Upon receiving information that a monitored individual is a confirmed carrier of a contagious disease (box 420), the system can determine the probability that the owners of other devices (e.g., P2, FIG. 1C) have contracted the contagious disease (box 430). If the calculated probability is larger than a predetermined value p≥x (box 440), the system can inform a relevant entity (e.g., the possibly infected person, a healthcare authority, etc.) that the owner of the other device (e.g., P2, FIG. 1C) may have been infected (box 450). The system can then continue to assess the contacts made by the second person (P2) for determining probability of having contracted the contagious disease.

In addition to determining probability of infection, embodiments disclosed herein can be utilized for ensuring quarantine adherence, for example by identifying whether individuals under quarantine and/or individuals under “stay at home” restrictions are remaining compliant. For example, during COVID-19 “stay at home” restrictions, States advised that if those deemed as an “essential business” should limit their contact with other individuals to a certain range, namely six feet. In addition, individuals who have not been deemed “essential” were told that they could leave their homes to buy groceries, gas and engage in other essential activities. As they do this, they were advised to also maintain six feet of distance between other individuals. Embodiments disclosed herein can be used to ensure compliance with such directives, and in more severe circumstances, send alerts to both the individual and also to public health/law enforcement, in the event that individuals are coming close to violating the six feet rule.

Additionally or alternatively, embodiments disclosed herein can be utilized for ensuring adherence to restraining orders. For example, embodiments disclosed herein can be used to ensure that individuals that have been served a restraining order are compliant with the terms of that restraining order. In addition, proactive and reactive alerts can be sent to law enforcement in the event the restraining order is violated or about to be violated. For example, limits could be set so that law enforcement is notified if an individual comes within 1,000 feet of another individual. These same proactive and reactive alerts may also be sent to the individual who has filed the restraining order.

Additionally or alternatively, embodiments disclosed herein can be utilized to track the location of a suspected Amber Alert victim or assailant. In certain cases, GPS tracking may not be available to use. In such cases, by scanning all mobile phones in a particular area to determine whether they have (e.g., via Wi-FI, Bluetooth and Near Field Communication) passively communicated with an assailant or victims phone, embodiments disclosed herein can trace/re-trace the steps of a victim and assailant to determine their location and/or direction of movement.

Further, embodiments disclosed herein can be utilized to better ascertain the location of a 911 caller. In the event the individual cannot be traced by the 911 operator (for example, because the call is disconnected prematurely and/or because there is no ability to identify location through GPS), this technology could be used to determine the last set of individuals the 911 caller came into close contact with as an alternative means to determine their location. Similarly, embodiments disclosed herein can be used by law enforcement to determine the location of a person of interest.

While the invention has been particularly shown and described with reference to specific illustrative embodiments, it should be understood that various changes in form and detail may be made without departing from the spirit and scope of the invention.

1. A system for assessment of probability of disease transmission, the system comprising:
at least one memory operable to store content collected from one or more user devices over a predetermined period of time; and
at least one processor communicatively coupled to the at least one memory, the processor being operable to:
analyze content received from the one or more user devices to determine whether a device belongs to an infected user having a contagious disease; and
in an event the device belonging to the infected user having the contagious disease is identified, obtain information regarding user devices which have been in a predetermined proximity of the infected user;
analyze the information to determine a probability that a user of each of the user devices which have been in the predetermined proximity of the infected user has contracted the contagious disease; and
in an event the probability that the user has contracted the contagious disease exceeds a predetermined threshold, transmit information to the user and/or public health officials regarding possibility of infection,
wherein the user devices which have been in the predetermined proximity of the infected user are identified based on global positioning system (GPS) sensor disposed in the user devices, and
wherein the information to determine the probability includes whether or not contagions have been released by the infected user at a specific location and a specific time.

2. The system of claim 1, wherein the contagious disease comprises an airborne disease.

3. The system of claim 1, wherein the at least one processor is further operable to inform at least one third party of the probability that the user has contracted the contagious disease.

4. A system for assessment of probability of disease transmission, the system comprising:
at least one memory operable to store content collected from one or more communications devices over a predetermined period of time; and
at least one processor communicatively coupled to the at least one memory, the processor being operable to:
collect information indicating whether a first individual carrying a first communications device among the one or more communication devices has been within a predetermined range of one or more individuals carrying respective communications devices;
receive information that the first individual is a confirmed carrier of a contagious disease;
determine a probability that at least one individual from the one or more individuals has contracted the contagious disease; and
transmit information to the at least one individual regarding the probability that they have contracted the contagious disease;
wherein each of the one or more communication devices comprises a global positioning system (GPS) sensor to collect location information therewith, and
wherein the probability is determined based on whether or not contagions have been released by the first individual at a specific location and a specific time.

5. The system of claim 4, wherein the predetermined range comprises a range in which contagions released from body of the first individual are dispersed.

6. The system of claim 4, wherein whether or not the contagions have been released by the first individual is determined using at least one of a motion sensor or an audio sensor disposed in the first communication device belonging to the first individual.

7. The system of claim 6, wherein the motion sensor is configured to detect a motion of an arm of the first individual in response to sneezing or covering cough or sneeze, and
wherein the audio sensor is configured to detect a sound associated with coughing or sneezing.

8. The system of claim 6, wherein the first communication device comprises at least one of a thermometer and a heart rate monitor.

9. The system of claim 8, wherein the processor is further operable to confirm whether the first individual is a confirmed carrier of the contagious disease based on data collected using the at least one of the thermometer and the heart rate monitor.

10. The system of claim 4, wherein the one or more communications devices comprise mobile phones.

11. The system of claim 4, wherein the range is at least six feet.

12. The system of claim 4, wherein the processor is configured to determine the probability as a function of number of times that the at least one individual has been within the predetermined range of the first individual.

13. The system of claim 4, wherein the processor is configured to determine the probability as a function of a length of time that the at least one individual has been within the predetermined range of the first individual.

14. The system of claim 4, wherein the predetermined period of time is at least fourteen days.

15. The system of claim 4, wherein information collected from the first individual comprises at least one of a phone number of the communications device carried by the first individual and a geographical location of the first individual.

16. The system of claim 4, wherein information collected from the one or more individuals comprises at least one of a phone number of the communications device carried by each individual and a geographical location of each individual.

17. The system of claim 4, wherein the contagious disease comprises an airborne disease.

18. The system of claim 4, wherein the processor is configured to determine a distance between the first individual and each of the one or more individuals and record at least some information regarding the respective communications devices of the one or more individuals in an event the distance is within the predetermined range.

19. The system of claim 4, wherein the processor is configured to receive the information that the first individual is a confirmed carrier of a contagious disease from at least one of: the first individual and a health authority monitoring the first individual.

20. A system for assessment of probability of disease transmission, the system comprising:
a global positioning system (GPS) sensor;
at least one of a motion sensor or an audio sensor; and
at least one processor communicatively coupled to the GPS sensor and the at least one of the motion sensor or the audio sensor, the processor being operable to:
collect location information with respect to time using the GPS sensor;
transmit information that a user of the system is a confirmed carrier of a contagious disease;
transmit information to determine a probability that contagions have been released at a specific location and a specific time;
wherein the information to determine the probability that contagions have been released at the specific location and the specific time includes data collected using the at least one of the motion sensor or the audio sensor, and the location information collected using the GPS sensor.

21. The system of claim 20, wherein the motion sensor is configured to detect a motion of an arm of the user in response to sneezing or covering cough or sneeze, and
wherein the audio sensor is configured to detect a sound associated with coughing or sneezing.

22. The system of claim 20, wherein the system is implemented as a mobile device.

23. The system of claim 20, wherein the system is implemented as a wearable device.

24. The system of claim 1, wherein whether or not the contagions have been released by the infected user is determined using the at least one of a motion sensor or an audio sensor disposed in the device belonging to the infected user.

25. The system of claim 24, wherein the motion sensor is configured to detect a motion of an arm of the infected user in response to sneezing or covering cough or sneeze, and
wherein the audio sensor is configured to detect a sound associated with coughing or sneezing.
